(19) United States
(12) Patent Application Publication
(10) Pub. No.: US 2003/0203019 A1
(43) Pub. Date: Oct. 30, 2003

(54) Coated conditioners for use in foods and pharmaceuticals

(76) Inventors: John Mark Cornelius, Forest Hill, MD (US); Michael E. Tarquini, Havre de Grace, MD (US); June M. Neades, Havre de Grace, MD (US); Gary M. Freeman, Macon, GA (US)

Correspondence Address: David Mitchell Goodrich, Esq., J. M. Huber Corporation, 333 Thornall Street, Edison, NJ, 08837-2220, US

(21) Appl. No.: 10/135,978

(22) Filed: Apr. 30, 2002

(51) Int. Cl. A61K 9/20; A61K 31/16

(52) U.S. Cl. 424/465; 514/629

(57) ABSTRACT

An edible composition comprising a coated conditioner that contains a hydrophobization agent and inorganic particles is provided. When incorporated into an edible composition (such as a powdered pharmaceutical or food product), the coated conditioners inhibit caking and promote the free flow of powder. Suitable hydrophobization agents include food-grade fatty acids, food-grade oils, food-grade waxes, and food-grade gums, while suitable inorganic particles are selected from the group consisting of silica, silicates, calcium carbonates, phosphates, and clays. The coated conditioner is particularly suitable for use in pharmaceutical preparations, such as acetaminophen.

### BACKGROUND OF THE INVENTION

For the last several years materials such as silica, sodium aluminosilicates, kaolin clays, tricalcium phosphate, and calcium silicates have been used as “conditioners” in dry and powdered foods to prevent caking and encourage the free flow of powdered food particles. In pharmaceuticals, fumed silica has been widely used as an excipient (conditioner or glidant) for the same reasons. These conditioners absorb moisture from the atmosphere or package to prevent the food particles from sticking together in moisture or pressure cakes and also act as “ball bearings” to coat the surface of the food particles, thus preventing agglomeration among adjacent particles. These conditioners, also known as free flow, and anticaking agents are permitted for use at levels less than or equal to 2.0 wt % in the final food product by the U.S. Food and Drug Administration. Additionally these conditioners may also be used in other applications such as fertilizers, pesticides, and polymers.

While these conditioners are used in many commercially-prepared food powders susceptible to pressure or moisture caking, they lack efficacy for use in many pharmaceuticals, as well as certain food products that are hygroscopic, contain high concentrations of proteinaceous material, or have a high content of fats and oils such as garlic powder, de-lactosed milk powder or hydrolyzed vegetable powder. In fact, for many foods and pharmaceuticals a suitable conditioner is not available. Certain materials, such as the J. M. Huber Corporation's Zeosyl® T 166 (a silica treated with a siloxane to render the silica hydrophobic) can significantly inhibit caking in foods and pharmaceuticals. However, silane-treated silicas are only permitted in food applications as defoaming agents for beet and cane sugar. They are not permitted for use as food conditioners.

Thus for many pharmaceuticals and food products there is no approved commercially-available conditioner that provides excellent anti-caking performance. For example, the common pain reliever acetaminophen (N-acetyl-para-aminophenol) has a tightly packed crystalline form that often results in the formation of pressure and moisture cakes of the powder during storage, leading to poor flow performance. Commercially-available fumed silicas, such as Cab-O-Sil® M5 from the Cabot Corporation, Bellrica, Mass., provide some improvement in flow performance, but they do not completely address the problem.

Given the forgoing there is a continuing need for chemical conditioners suitable for use in certain pharmaceuticals and food products that provide excellent anti-caking properties to ensure good flow performance, while at the same time present no health or safety concerns that would prohibit their use by food safety regulatory authorities.

### BRIEF SUMMARY OF THE INVENTION

The invention includes an edible composition comprising a coated conditioner, the conditioner containing a hydrophobization agent and inorganic particles

The invention also includes a pharmaceutical preparation comprising a pharmaceutically active ingredient and a coated conditioner, the conditioner containing inorganic particles and a hydrophobization agent.

The invention also includes an acetaminophen pharmaceutical preparation comprising acetaminophen, and a coated conditioner comprising (i) inorganic particles; and (ii) 1 wt % to about 20 wt %, based on the total weight of the conditioner, of an hydrophobization agent.

### DETAILED DESCRIPTION OF THE INVENTION

All parts, percentages and ratios used herein are expressed by weight unless otherwise specified. All documents cited herein are incorporated by reference.

By “mixture” it is meant any combination of two or more substances, in the form of, for example without intending to be limiting, a heterogeneous mixture, a suspension, a solution, a sol, a gel, a dispersion, or an emulsion.

By “coated” it is meant that the specified coating ingredient covers at least a portion of the outer surface of a particle or substrate.

By “inorganic particulates” it is meant both naturally occurring inorganic minerals and synthetically produced inorganic compounds.

By “food product” it is meant any product meant to be consumed, as well as additives to food products such as, without intending to be limiting, spices, seasonings, food colorants, anti-caking and free flow agents.

The present invention relates to coated conditioners that when incorporated into powdered pharmaceutical or food products inhibit caking and promote the free flow of the powder. These coated conditioners are a mixture of a hydrophobization agent (such as a stearic compound or an oil) and well-known inorganic particulates such as kaolin clay, silica, silicates, phosphates, and calcium carbonates. These coated conditioners are not only functionally effective, but because the conditioners are merely a mixture of two components (bydrophobization agent and inorganic particulates) that have previously been approved food additives, then the conditioners are safe for use in pharmaceuticals and food products.

The ingredients of the coated conditioner as well as a method for making the coated conditioner will now be discussed in detail. Then powdered pharmaceutical or food products that make use of the coated conditioners will be discussed and examples of such products provided.

The coated conditioners prepared according to the present invention are composed of at least two components: inorganic particles and hydrophobizing compounds. The inorganic particles are selected from any inorganic compounds commonly used as conditioners in food and pharmaceutical powders, such as silica (such as precipitated silica or fumed silica and silica gel), precipitated or ground calcium carbonates, kaolin clays, silicates (such as calcium silicate, magnesium silicate, aluminum calcium silicate, tricalcium silicate, sodium calcium aluminosilicate, sodium magnesium aluminosilicate, and sodium aluminosilicate) and phosphates (such as tricalcium phosphate, dicalcium phosphate, monocalcium phosphate, magnesium phosphate). Preferably, the inorganic particles serve as substrates, i.e., the inorganic particles are coated with the hydrophobization agent.

The preferred silicas are amorphous precipitated silicas that are produced from a liquid phase by acidulating an alkali metal silicate with a strong acid such as sulfuric acid, in the presence of heat. Useful techniques for conducting the precipitation (acidulation) reaction itself to produce homogenous amorphous silica particles are widely known and understood. The resulting silica precipitate is filtered, washed, and dried in manners such as customarily practiced. Examples of the many patented publications describing such precipitated silicas include U.S. Pat. Nos. 4,122,161, 5,279,815 and 5,676,932 to Wason et al., and U.S. Pat. Nos. 5,869,028 and 5,981,421 to McGill et al.

After being produced by the aforementioned liquid phase method, the precipitated silica may then be milled to obtain the desired particle size of between about 4 μm to 25 μm, such as about 4 μm to about 15 μm. Said silicas will preferably have oil absorption of about 50 ml/100 g to about 475 ml/100 g. Suitable silicas are manufactured by the J.M. Huber Corporation, Edison, N.J., and are sold in different grades under the trademarks Zeofree®, Zeosyl® and Zeothix®.

Synthetic amorphous alkaline earth metal silicates, such as amorphous calcium silicate, may also be used as the inorganic particles. These silicates are most typically prepared by the reaction of a reactive silica with an alkaline earth metal reactant, preferably an alkaline earth metal oxide or hydroxide, and a source of aluminum such as sodium aluminate or alumina. Because the final properties of the silicate are dependent on the reactivity of the silica, the silica source is preferred to be a clay which has been treated with a mineral acid (such as sulfuric acid) to produce alum (aluminum sulfate) and an insoluble reactive silica. A suitable example of this is sulfuric acid leached reactive clay. Suitable synthetic amorphous alkaline earth metal silicates are manufactured by the J.M. Huber Corporation and are sold in different grades under the trademark Hubersorb® Methods and techniques for preparing these silicas are discussed in greater detail in U.S. Pat. No. 4,557,916. Other suitable silicates are available from J.M. Huber Corporation such as sodium aluminosilicate sold under the trademark Zeolex® and sodium magnesium aluminosilicate sold under the trademark Hydrex®.

Also suitable for use as inorganic particles are ground calcium carbonate or precipitated calcium carbonate. Ground calcium carbonate is first mined and then ground to the appropriate particle size. Optionally, ground calcium carbonate may be classified into more narrow particle size fractions. Precipitated calcium carbonate is typically obtained by exposing calcium hydroxide slurry (i.e., milk of lime) to a carbonation reaction. This may be done by injecting carbon dioxide gas into a reaction vessel containing aqueous calcium hydroxide slurry. Methods and techniques for preparing these precipitated calcium carbonates are discussed in greater detail in U.S. Pat. No. 4,888,160. Suitable precipitated calcium carbonates are manufactured by the J.M. Huber Corporation and are sold in different grades under the trademark HuberCal®.

Also suitable for use as inorganic particles are clays such as kaolin clays. These clays are produced by first mining raw clay, and then subjecting the mined clay to several beneficiating steps until it is suitable for use in a consumer product. The beneficiating steps include, for example: removing grit particles, sorting the clay particles to obtain a more desirable particle size distribution; removing several different impurities found in the raw clay, and steps to impart to the clay a more desirable final color. Suitable kaolin clays are manufactured by the J.M. Huber Corporation and are sold in different grades under the trademark Polygloss®.

Hydrophobization agents include food-grade fatty acids, particularly stearic compounds, food-grade oils, and food-grade waxes and gums. Suitable fatty acids include capric, capryllic, lauric, myristic, oleic, palmitic and stearic acids, as well as the fatty acid compounds listed in Title 21 C.F.R. (the United States' Code of Federal Regulations) as permitted for direct addition to food, feed or pharmaceuticals. Suitable stearic compounds include stearic acids, salts of stearic acid and esters of stearic acid. Suitable salts of stearic acid include magnesium stearate, calcium stearate, potassium stearate and zinc stearate. A suitable magnesium stearate is the vegetable-based, food grade magnesium stearate available from Ferro Chemicals, Cleveland, Ohio under the Synpro® trademark. Suitable esters of stearic acid include alcohol stearic acid esters such as glycerylmonostearate and triglyceryl stearate. Suitable esters of stearic acid include alcohol stearic acid esters such as glyceryl monostearate and glyceryl tristearate, as well as other esters such as glyceryl palmitostearate, and sorbitan monostearate. Glyceryl monostearate and glyceryl tristearate are available from Patco Corporation, Wilmington, Del. under the trademarks Pationic® 901 and Pationic® 919, respectively.

Food-grade oils are those oils listed in 21 C.F.R. as permitted for direct addition to food, feed or pharmaceuticals. Suitable food-grade oils include white mineral oil, rapeseed oil, soybean oil, castor oil, coconut oil and oils defined as “essential oils” by the F.D.A. in 21 C.F.R. §182.20. Suitable food-grade waxes and gums may also be located in 21 C.F.R. Suitable food-grade waxes include candelilla, carnuba and paraffin waxes. Suitable food-grade gums include karaya gum, gum tragacanth, carrageenan gum, xanthan gum, and guar gum.

A preferred process for combining the aforementioned ingredients to form a coated conditioner (in which the hydrophobization agents are stearic compounds) can be summarized as follows. In a first step of this process, an amount of the inorganic particles is added to a mixing bowl and preferably heated to a temperature of 10° F. to 30° F. above the melting point of the stearic compound. The rotating blades of the mixer are turned on, a stearic compound added to the bowl. Mixing continues for about 30 minutes. After mixing is completed, the material inside the mixing bowl (the coated conditioner) is allowed to cool before it is added to a powdered food or pharmaceutical product. When the hydrophobization agent is an oil, an identical process as that described is followed with the exception that no heating is required, because the oils are liquid at ambient temperature.

The invention will now be described in more detail with respect to the following, specific, non-limiting examples.

### EXAMPLES

In Examples 1-8, coated conditioners that are a mixture of a stearic compound and inorganic particles were prepared according to the present invention. The inorganic particles have median particle size and oil absorption values given in Table A, below (methods for determining particle size and oil absorption value are discussed below).

In this process, first, an amount of inorganic particles, such as silica, calcium carbonate or kaolin clay (as indicated in Table I below) was added to a mixing bowl and the mixing bowl attached to a Kitchen Aid Heavy Duty mixer, model K5SS. To control the temperature of the mixing bowl contents, a thermal jacket was wrapped around it so as to heat the mixing bowl to increase the temperature of the mixing bowl contents. The temperature of its contents were measured by a thermocouple placed in contact with the mixing bowl contents, and the temperature was electronically regulated through a solid state temperature controller connected to both the thermocouple and the thermal jacket.

The temperature controller was set at 177° C. and the mixer turned on low. After the temperature of the inorganic particles in the mixing bowl reached 177° C., powdered magnesium stearate (vegetable-based, food grade magnesium stearate available from Ferro Chemicals, Cleveland, Ohio under the Synpro® trademark) was added to the inorganic particles in the mixing bowl in the weight proportions set forth in Table 1 below and mixing was allowed to continue for 10 minutes. The wt % of magnesium stearate added is based on the total weight of the coated conditioner (i.e., the weight of the inorganic substrate plus the weight of magnesium stearate). Following mixing the resulting coated conditioner powder was allowed to cool to ambient temperature. During mixing, the magnesium stearate was melted onto the particulate inorganic substrate so that the coated conditioner was a mixture of magnesium stearate and the particulate mineral substrate.

The particle size and the oil absorption of the inorganic particles used in the following examples are as follows:

TABLE A

| | Median Particle Size | Oil Absorption |
|---|---|---|
| Inorganic Particle | μm | mL/100 g |
| Zeofree 80 silica | 14 | 195 |
| Zeothix 265 silica | 4 | 220 |
| HuberCal 250 GCC | 14 | 13 |
| Hubersorb 600 calcium silicate | 6 | 475 |
| Polygloss 90 clay | 0.4 | 42 |

Zeofree® 80 and Zeothix® 265 amorphous precipitated silicas are available from the J.M. Huber Corporation. Polygloss® 90, a kaolin clay, and HuberCal™ 250, a ground calcium carbonate, are both available from the J.M. Huber Corporation. Hubersorb® 600 is a calcium silicate from the J.M. Huber Corporation

The oil absorption was measured using linseed oil by the rubout method. In this test, oil is mixed with a silica and rubbed with a spatula on a smooth surface until a stiff putty-like paste is formed. By measuring the quantity of oil required to have a paste mixture, which will curl when spread out, one can calculate the oil absorption value of the silica—the value which represents the volume of oil required per unit weight of silica to completely saturate the silica sorptive capacity. Calculation of the oil absorption value was done according to equation (I):

$$\begin{matrix}Oil absorption = \frac{ml oil absorbed}{weight of silica , grams} \times 100 \\ = ml oil \text{/} 100 gram silica\end{matrix} \qquad ( I )$$

The particle size was determined using a Model LA-910 laser light scattering instrument available from Horiba Instruments, Boothwyn, Pa. A laser beam is projected through a transparent cell which contains a stream of moving particles suspended in a liquid. Light rays which strike the particles are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution.

The compositions of the coated conditioners of example numbers 1-8 are as follows:

TABLE I

| | Inorganic | Wt % of Magnesium |
|---|---|---|
| Example No | Substrate | Stearate |
| 1 | Zeofree 80 | 2 |
| 2 | Zeofree 80 | 4 |
| 3 | Zeothix 265 | 2 |
| 4 | Zeothix 265 | 4 |
| 5 | Polygloss 90 | 2 |
| 6 | Polygloss 90 | 4 |
| 7 | HuberCal 250 | 2 |
| 8 | HuberCal 250 | 4 |

Further coated conditioner samples (Examples 9-25) were prepared in a manner similar to that set forth above for examples 1-8, except that a Thysson Henschel FM 100 mixer was used. The participation inorganic substrates, as indicated in Table II below, were placed in a mixing bowl attached to the mixer and preheated to 77° C. while the mixing blade rotated at 860 rpm. After reaching 77° C., an amount of stearate, as indicated in table II below, is added. The inorganic particles and the stearate are allowed to mix at 77° C. for 10 minutes. The exception is that when magnesium stearate is used, the temperature is set to 170° C. Following mixing the resulting coated conditioner powder is allowed to cool to ambient temperature.

The compositions of the coated conditioners of example numbers 9-25 are as follows:

TABLE II

| Example | Inorganic | | Weight % of |
|---|---|---|---|
| No | Substrate | Stearate | Glyceryl stearate |
| 9 | Zeofree 80 | Glyceryl monostearate | 2 |
| 10 | Zeofree 80 | Glyceryl monostearate | 4 |
| 11 | Zeofree 80 | Glyceryl tristearate | 2 |
| 12 | Zeofree 80 | Glycery tristearate | 4 |
| 13 | Zeothix 265 | Glyceryl monostearate | 2 |
| 14 | Zeothix 265 | Glyceryl monostearate | 4 |
| 15 | Zeothix 265 | Glyceryl tristearate | 2 |
| 16 | Zeothix 265 | Glyceryl tristearate | 4 |
| 17 | Polygloss 90 | Glyceryl monostearate | 2 |
| 18 | Polygloss 90 | Glyceryl monostearate | 4 |
| 19 | Polygloss 90 | Glyceryl tristearate | 2 |
| 20 | Polygloss 90 | Glyceryl tristearate | 4 |
| 21 | HuberCal 250 | Glyceryl monostearate | 2 |
| 22 | HuberCal 250 | Glyceryl monostearate | 4 |
| 23 | HuberCal 250 | Glyceryl tristearate | 2 |
| 24 | HuberCal 250 | Glyceryl tristearate | 4 |
| 25 | Hubersorb 600 | Magnesium Stearate | 4 |

Hubersorb 600 calcium silicate is available from the J.M. Huber Corporation. The wt % of the stearate added is based on the total weight of the coated conditioner (weight of mineral substrate plus weight of the stearate).

In Examples 26-29, coated conditioners that are a mixture of mineral oil and precipitated silica were prepared according to the present invention. First, 100 grams of precipitated silica (as indicated in Table III, below) was added to a mixing bowl and the mixing bowl attached to a laboratory-scale Hobart mixer. The mixer was turned on low speed and at ambient temperature 4.0% or 10.0% of mineral oil was added and allowed to mix with the silica for 10 minutes (as indicated in Table III, below).

TABLE III

| | Inorganic | Mineral Oil |
|---|---|---|
| Example Number | Substrate | Addition |
| 26 | Zeofree 80 | 4% |
| 27 | Zeofree 80 | 10% |
| 28 | Zeothix 265 | 4% |
| 29 | Zeothix 265 | 10% |

The likelihood of a powder to form a moisture cake was evaluated using the moisture caking test. Before actual testing of the conditioned powder in the moisture cake itself (i.e., pharmaceutical or food powder containing a conditioner) was done, a baseline correlation between moisture and caking for unconditioned pharmaceutical or food powder was determined. To established this correlation, acetaminophen powder is titrated with a minimum amount of water to produce near 0% caking, and also an acetaminophen powder is titrated with a maximum amount of water to produce 70-80% caking. These points are then plotted along a straight line, and the amount of water needed to produce about 50% caking is the amount then used for the remaining conditioned acetaminophen powder samples to be tested.

The above moisture measurements were carried out as follows. A sufficient amount of screened, unconditioned sample was placed into an 8 oz. Spex® Mill jar so that the jar was about one-half full. Either 1 ml or 1 g of water was titrated or weighed onto the sample in the jar, and then the jar and its contents were placed on the Spex Mill (model 8000-115 available from Spex Corporation, Edison, N.J.) for 30 seconds. Then a small aluminum pan was prepared for use by pressing the lid of the Spex Jar to the bottom of the pan to mold the contour of the pan to the shape of the lid. 20 g of wet sample was then weighed onto the pan. Then from this 20 g of sample a level cake was formed by placing a jar filled with lead shot, lid side down, on each sample. Samples were then placed in an oven for at least 15 minutes at 50° C. to expel the added moisture and set the cake. Sample weight should be checked to confirm that all of the added water has been driven off. Longer times or higher temperatures may be required to remove all of the water. As the testing is done in triplicate, three jars are prepared for each sample component.

the samples were then removed from the oven and allowed to cool for ten minutes to room temperature. If the samples are not allowed to cool to room temperature, artificially low % moisture caking will result. The samples were not allowed to cool more than 15 minutes (because once they are cooled, samples can begin to absorb moisture, which can soften the cake and result in an artificially low % moisture cake).

Next, a #12 Tyler screen was inverted and centered over each aluminum pan, and the aluminum pan held against the # 12 screen, at the same time that the sample was carefully inverted over the screen so that the cake comes to rest on the #12 screen as the aluminum pan is removed. The screen was then transferred to a Thomas orbital sieve shaker (available from Thomas Scientific Apparatus) without breakage, and the caked sample vibrated on the Thomas Shaker for one minute. The amount of sample remaining on the screen is weighed, and percent cake is calculated as in equation (II):

$$\frac{grams of cake left on screen}{( x - \frac{xy}{z + y} )} * 100 = \% cake \qquad ( II )$$

wherein:

x=g of sample used in aluminum pan

y=ml H₂O added to sample in jar

z=g of sample added to jar

After the percent cake has been determined in triplicate for 1 ml of added water, then the process described above was repeated using 2 ml of water, then 3 ml, etc., until 80% caking is reached. Products, which are very sensitive to moisture may require increments of water below 1 ml. Water addition levels should be adjusted until there are at least four data points on the caking curve between 10-80%. The results of the % cake test for garlic powder and acetaminophen are set forth below.

To measure the loose bulk density, a modified 250-mL graduated cylinder is utilized. The cylinder has been modified such that the cylinder top is level with the 1100 ml mark, by cutting off the cylinder at the 100-mL mark. The empty cylinder weight is recorded as the “tare weight”. The sample powder was poured into the modified cylinder until overflowing. The level of powder in the cylinder was immediately leveled-off by scraping across the top with a spatula; this leveling-off step was done as quickly as possible to prevent settling of the powder, which would give artificially high loose bulk density values. Any additional excess powder along the sides or base of the graduated cylinder was also brushed off and the cylinder weighed, with the weight recorded as the “total weight”. Any volume change noticed after the powder has been leveled off and excess powder brushed away is to be ignored, because this volume change is due to the tendency of the powder to pack down. The loose bulk density is calculated from equation II:

$$Loose Bulk Density , g \text{/} ml = \frac{total wt . \text{-} tare wt .}{100} \qquad ( II )$$

Another useful measure of powder flowability is the avalanche time, which is measured as the “Aeroflow parameter”. The shorter the time between avalanches, the more free flowing the powder. In this test, first an amount of unconditioned sample was used to determine the weight needed to fill a 100 ml graduated cylinder, as in the loose bulk density test, above. This weight was then used for all runs of the Aeroflow® tests. An Aeroflow® Powder Flowability Analyzer Model 0-8030 from TSI Incorporated of St. Paul, Minn. was used in these tests. In the first step of these tests, a ring of masking tape was applied to the inner surface of an Aeroflow testing drum. The masking tape acts, in effect, as a gasket to prevent the powder from leaking during operation. The powder sample was then loaded into the drum and the drum placed into the Aeroflow testing device. Using the computer interface, the Aeroflow test was selected, and the Hardware Configuration settings on the instrument checked to verify that the drum speed is 60 rpm. The “apply” feature was then selected and the drum allowed to rotate for 5 minutes. After 5 minutes the device was manually stopped by the operator by depressing the “Close” button. Then the mean avalanche time was determined with the drum speed set at 60 rpm and the test duration set at 300 seconds. For each sample, the test was repeated one additional time and the results averaged and expressed in seconds.

Also measured was the Flowdex parameter. Flowdex is a measure of flowability that simulates the flowability of a powder in a silo. 25 g of a sample is placed in a funnel, which is placed above the Flowdex straight walled, open cylinder. In the bottom of the cylinder is a plate with an opening of known diameter. Several different plates with different diameter openings (“orifices”) are available, so the plates can be interchanged until the minimum opening required for the sample to flow through is determined. The smaller the opening required for a given material to flow through, the more easily the material will flow in a bag house or silo.

In the test, the Flowdex apparatus (model 21-100-004 available from Hanson Research, Chatsworth, Calif.) is prepared in accordance with the manufacturer's instructions with the smallest orifice supplied by the manufacturer installed (and a removable stopper installed under the orifice). 25.00 g of the sample is weighed and poured into the upper funnel, and the timer started. After 30 seconds, the stopper is removed from the orifice, and the sample allowed to flow (if possible) though the orifice. The device is inspected to determine if the sample flowed through the orifice such that the bottom of the instrument is visible, if the bottom of the instrument is visible, the orifice diameter is recorded, and this is taken as the Flowdex value. If the bottom is not visible, the orifice is replaced with the next larger size and the above procedure repeated. Once an orifice has been found that permits flow, the test is repeated with the same orifice to confirm the orifice diameter value.

To demonstrate their efficacy in consumer products, coated conditioners prepared according to Examples 1-24 were incorporated into acetaminophen powder compositions at three different concentration levels, 0.1 wt %, 0.5 wt %, 1.0 wt %. As a control the most widely used conditioner for acetaminophen, Cab-O-Sil® M5 fumed silica, was added to a separate acetaminophen composition. As discussed above, acetaminophen has a tightly packed crystalline form that often results in the formation of pressure and moisture cakes of the powder during storage.

The percent cake, loose bulk density, the Aeroflow parameter and the Flowdex parameter were measured for acetaminophen and the Results are set forth in tables IV-VII, below:

TABLE IV
Moisture Caking of Acetaminophen

| | Concentration of | | |
|---|---|---|---|
| Coated | Conditioner (in Wt %) | | |
| Conditioner | 0.1 | 0.5 | 1.0 |
| Unconditioned | 46.0 | 46.0 | 46.0 |
| Control Conditioner: | 61.8 | 85.7 | 85.0 |
| (Cab-O-Sil ® M5) | | | |
| Example 1 | 50.9 | 75.8 | 80.8 |
| Example 2 | 58.1 | 78.7 | 79.6 |
| Example 3 | 52.1 | 83.4 | — |
| Example 4 | — | 81.9 | 85.4 |
| Example 5 | 45.0 | 63.4 | 85.9 |
| Example 7 | 47.8 | 46.0 | 52.0 |
| Example 8 | 44.2 | 48.6 | 49.5 |
| Example 9 | 52.1 | 79.1 | 84.7 |
| Example 10 | 62.2 | 71.0 | 52.0 |
| Example 11 | 71.2 | 65.2 | 79.0 |
| Example 12 | 55.4 | 83.0 | 67.4 |
| Example 13 | 65.8 | 12.4 | 80.3 |
| Example 14 | 63.2 | 81.9 | 69.4 |
| Example 15 | 65.8 | 64.7 | 80.3 |
| Example 16 | 46.1 | — | 68.9 |
| Example 17 | 50.8 | 13.6 | 91.1 |
| Example 18 | 52.1 | 78.2 | 89.5 |
| Example 19 | 50.4 | 53.9 | 72.8 |
| Example 20 | 47.6 | 54.8 | 74.4 |
| Example 21 | 49.4 | 42.4 | 42.2 |
| Example 22 | 40.8 | 34.9 | 38.5 |
| Example 23 | 47.3 | 46.3 | 43.2 |
| Example 24 | 45.8 | 42.8 | 47.5 |

Moisture caking is not a significant problem for unconditioned acetaminophen powder. However, it is important that conditioner added to acetaminophen to improve flow not be deleterious to moisture caking. It is seen from the above data that the industry standard used to improve flow of acetaminophen (Cab-O-Sil M5) actually is detrimental to moisture caking, while most of the inventive example conditioners perform better than Cab-O-Sil M5 and some actually improve moisture caking. Tables V and VI below show the flow properties of these same conditioners.

TABLE V
Aeroflow Parameter of Acetaminophen

| | Concentration of | | |
|---|---|---|---|
| Coated | Conditioner (in Wt %) | | |
| Conditioner: | 0.1 | 0.5 | 1.0 |
| Unconditioned | 3.00 | 3.00 | 3.00 |
| Control Conditioner: | 1.86 | 1.88 | 2.02 |
| (Cab-O-Sil M5) | | | |
| Example 1 | 1.67 | 1.70 | 1.91 |
| Example 2 | 1.82 | 1.79 | 1.86 |
| Example 3 | 1.72 | 1.83 | 1.89 |
| Example 4 | 1.81 | 1.81 | 1.92 |
| Example 5 | — | 2.25 | 2.13 |
| Example 6 | 2.09 | 2.27 | 2.15 |
| Example 7 | 2.77 | 2.74 | 3.05 |
| Example 8 | 3.06 | 2.98 | 2.77 |
| Example 9 | 1.85 | 1.74 | 1.99 |
| Example 10 | 1.78 | 1.72 | 1.94 |
| Example 11 | 1.84 | — | 1.96 |
| Example 12 | 1.80 | 1.78 | 1.97 |
| Example 13 | 1.75 | 1.85 | 1.97 |
| Example 14 | 1.73 | 1.85 | 1.87 |
| Example 15 | 1.77 | 1.78 | 2.06 |
| Example 16 | 1.84 | 1.94 | 2.06 |
| Example 17 | 2.68 | 2.49 | 2.02 |
| Example 18 | 2.17 | 2.30 | 2.20 |
| Example 19 | 2.10 | 2.27 | 2.00 |
| Example 20 | 2.33 | 2.25 | 2.02 |
| Example 21 | 2.77 | 2.80 | 2.85 |
| Example 22 | 3.31 | 3.24 | 3.11 |
| Example 23 | 2.72 | 2.70 | 2.87 |
| Example 24 | 3.08 | 2.85 | 2.91 |

As can be seen from the data in Table V, the acetaminophen powder incorporating coated conditioners prepared according to examples 1, 2, 3, 4, 9, 10, 11, 12, 13, and 14 had an improved measured Aeroflow Parameter (i.e., shorter avalanche times) when compared to acetaminophen powder incorporating the control fumed silica conditioner.

TABLE VI
Flowdex Parameter of Acetaminophen

| | Concentration of | | |
|---|---|---|---|
| | Conditioner (in Wt %) | | |
| Coated conditioner: | 0.1 | 0.5 | 1.0 |
| Unconditioned | 22 | 22 | 22 |
| Control Conditioner: | 4 | 5 | 7 |
| (Cab-O-Sil M5) | | | |
| Example 1 | 4 | 4 | 8 |
| Example 2 | 4 | 5 | 7 |
| Example 3 | 4 | 7 | 6 |
| Example 4 | 4 | 5 | 10 |
| Example 5 | 7 | 7 | 8 |
| Example 6 | 6 | 4 | 7 |
| Example 7 | 14 | 18 | 16 |
| Example 8 | 22 | 20 | 22 |
| Example 9 | 4 | 5 | 7 |
| Example 10 | 4 | 4 | 6 |
| Example 11 | 5 | — | 8 |
| Example 12 | 4 | 5 | 7 |
| Example 13 | 4 | 4 | 7 |
| Example 14 | 4 | 5 | 5 |
| Example 15 | 4 | 7 | 8 |
| Example 16 | 5 | 9 | 14 |
| Example 17 | 14 | 4 | 4 |
| Example 18 | 10 | 5 | 6 |
| Example 19 | 14 | 6 | 8 |
| Example 20 | 10 | 8 | 9 |
| Example 21 | 16 | 18 | 18 |
| Example 22 | 22 | 20 | 22 |
| Example 23 | 14 | 22 | 22 |
| Example 24 | 18 | 20 | 20 |

As can be seen in Table VI, all of the acetaminophen powders incorporating a coated conditioner prepared according to the present invention and using a silica as the inorganic particulate showed improved performance on the Flowdex test (i.e., passed through a narrower orifice) when compared to untreated acetaminophen powders. Many of the acetaminophen powders incorporating a coated conditioner prepared according to the present invention also showed improved performance on the Flowdex test when compared to acetaminophen powders incorporating the Cab-O-Sil product.

TABLE VII
Loose Bulk Density of Acetaminophen

| | Concentration of | | |
|---|---|---|---|
| | Conditioner (in Wt %) | | |
| Coated conditioner: | 0.1 | 0.5 | 1.0 |
| Unconditioned | 0.656 | 0.656 | 0.656 |
| Control Conditioner: | 0.699 | 0.674 | 0.645 |
| (Cab-O-Sil M5) | | | |
| Example 1 | 0.707 | 0.700 | 0.678 |
| Example 2 | 0.711 | 0.700 | 0.693 |
| Example 3 | 0.703 | 0.693 | 0.689 |
| Example 4 | 0.711 | 0.701 | 0.667 |
| Example 5 | 0.700 | 0.696 | 0.695 |
| Example 6 | 0.695 | 0.698 | 0.700 |
| Example 7 | 0.684 | 0.680 | 0.681 |
| Example 8 | 0.661 | 0.665 | 0.665 |
| Example 9 | 0.715 | 0.705 | 0.696 |
| Example 10 | 0.719 | 0.706 | 0.698 |
| Example 11 | 0.714 | 0.704 | |
| Example 12 | 0.727 | 0.714 | 0.699 |
| Example 13 | 0.742 | 0.728 | 0.702 |
| Example 14 | 0.733 | 0.712 | 0.699 |
| Example 15 | 0.711 | 0.693 | 0.662 |
| Example 16 | 0.724 | 0.708 | 0.673 |
| Example 17 | 0.690 | 0.699 | 0.686 |
| Example 18 | 0.708 | 0.711 | 0.697 |
| Example 19 | 0.705 | 0.710 | 0.689 |
| Example 20 | 0.717 | 0.710 | 0.700 |
| Example 21 | 0.660 | 0.667 | 0.662 |
| Example 22 | 0.659 | 0.654 | 0.651 |
| Example 23 | 0.665 | 0.668 | 0.681 |
| Example 24 | 0.655 | 0.650 | 0.659 |

Loose bulk density measurements generally show whether a material has been conditioned properly. A material that has been conditioned properly (i.e., that has maximum flow and minimum caking) will typically have an increased bulk density. Increased loose bulk density means that the product container package will not have to be enlarged when a conditioner is used. As can be seen in Table VII, several of the acetaminophen powders incorporating a coated conditioner prepared according to the present invention showed increased loose bulk density at all concentration levels when compared to the Cab-O-Sil product.

To demonstrate their efficacy in food products, coated conditioners prepared according to Examples 1-24 were incorporated into garlic powder compositions at three different concentration levels: 0.5 wt %, 1.0 wt %, 2.0 wt %. As a control, a widely used conditioner for food products, J.M. Huber's Zeofree 80, was also added to the garlic powder compositions.

TABLE VIII
% Moisture Caking in Garlic Powder

| | Concentration of | | |
|---|---|---|---|
| Coated | Conditioner (in Wt %) | | |
| Conditioner: | 0.5 | 1.5 | 2.00 |
| Unconditioned Garlic | 65.91 | 65.91 | 65.91 |
| Powder | | | |
| Example 1 | 60.31 | 24 | 14.1 |
| Example 2 | 56.62 | 21.01 | 13.5 |
| Example 3 | 44.62 | 12.31 | 5.88 |
| Example 9 | 53.7 | 34.92 | 20.7 |
| Example 12 | 60.73 | 24.28 | 20.2 |
| Example 27 | 62.68 | 37.98 | 25.2 |
| Example 29 | 53.67 | 23.96 | 12.5 |
| Control Conditioner: | 48.66 | 19.62 | 13.7 |
| Zeofree 80 | | | |

As is seen in from the data in Table VIII above, all of these conditioners, prepared in accordance with the invention, reduce the moisture caking of garlic powder. At the optimum treatment level of 2%, example 2, 3 and 29 conditioners performed better than the control conditioner.

TABLE IX
Aeroflow of Garlic Powder

| | Concentration of | | |
|---|---|---|---|
| Coated | Conditioner (in Wt %) | | |
| Conditioner: | 0.5 | 1.5 | 2.00 |
| Unconditioned Garlic | 2.93 | 2.93 | 2.93 |
| Powder | | | |
| Example 1 | 2.36 | 2.41 | 2.55 |
| Example 2 | 2.39 | 2.38 | 2.38 |
| Example 3 | 2.59 | 2.62 | 2.44 |
| Example 9 | 2.49 | 2.55 | 2.37 |
| Example 12 | 2.41 | 2.38 | 2.44 |
| Example 27 | 2.19 | 2.04 | 2.21 |
| Example 29 | 2.26 | 2.17 | 2.02 |
| Control Conditioner: | 2.56 | 2.58 | 2.52 |
| Zeofree 80 | | | |

As can be seen from the data in Table IX, the garlic powder incorporating coated conditioners prepared according to examples 1-3, 9, 12, 27, and 29 had an improved measured Aeroflow Parameter (i.e., shorter avalanche times) when compared to the garlic powder incorporating the control coated conditioner, Zeofree 80. Shorter avalanche times were not obtained with the coated conditioners prepared according to the other examples and so the results are not shown.

TABLE X
Flowdex of Garlic Powder

| | Concentration of | | |
|---|---|---|---|
| Coated | Conditioner (in Wt %) | | |
| Conditioner: | 0.5 | 1.5 | 2.00 |
| Unconditioned Garlic | 26 | 26 | 26 |
| Powder | | | |
| Example 1 | 28 | 28 | 28 |
| Example 2 | 24 | 26 | 24 |
| Example 3 | 28 | 28 | 30 |
| Example 9 | 24 | 24 | 26 |
| Example 12 | 26 | 26 | 26 |
| Example 27 | 28 | 26 | 24 |
| Example 29 | 26 | 26 | 26 |
| Control Conditioner: | 26 | 28 | 30 |
| Zeofree 80 | | | |

As can be seen in Table X, the garlic powders incorporating a coated conditioner prepared according to examples 2-3, 9, 12, 27, and 29 showed improved performance on the Flowdex test when compared to the garlic powder incorporating the control conditioner, Zeofree 80.

TABLE XI
Loose Bulk Density of Garlic Powder

| | Concentration of | | |
|---|---|---|---|
| Coated | Conditioner (in Wt %) | | |
| Conditioner: | 0.5 | 1.5 | 2.00 |
| Unconditioned Garlic | 0.496 | 0.496 | 0.496 |
| Powder | | | |
| Example 29 | 0.497 | 0.469 | 0.459 |
| Example 27 | 0.512 | 0.486 | 0.467 |
| Example 1 | 0.495 | 0.475 | 0.453 |
| Example 2 | 0.509 | 0.474 | 0.457 |
| Example 9 | 0.512 | 0.465 | 0.461 |
| Example 12 | 0.488 | 0.491 | 0.466 |
| Example 3 | 0.482 | 0.449 | 0.471 |
| Zeofree 80 | 0.513 | 0.479 | 0.47 |

The loose bulk density of garlic powder treated with conditioners 2, 9, and 27 at loading levels of 0.5% increased (improved). All of the conditioners decreased the garlic powder loose bulk density at higher loading levels.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An edible composition comprising a coated conditioner, the conditioner containing a hydrophobization agent and inorganic particles.

2. The edible composition according to claim 1, wherein the hydrophobization agent is selected from the group consisting of food-grade fatty acids, food-grade oils, food-grade waxes, and food-grade gums.

3. The edible composition according to claim 1, wherein the hydrophobization agent is a stearic compound.

4. The edible composition according to claim 1, wherein the hydrophobization agent is magnesium stearate.

5. The edible composition according to claim 1, wherein the hydrophobization agent is selected from the group consisting of stearic acid salts and stearic acid esters.

6. The edible composition according to claim 1, wherein the inorganic particles are selected from the group consisting of silica, silicates, calcium carbonates, phosphates, and clays.

7. The edible composition according to claim 1, wherein the conditioner comprises about 1 wt % to about 10 wt %, based on the total weight of the conditioner, of the hydrophobization agent.

8. The edible composition according to claim 1, wherein the hydrophobization agent is selected from the group consisting of glyceryl-monostearate and glyceryl tristearate.

9. The edible composition according to claim 1, wherein the composition is a powdered food product.

10. The edible composition of claim 1, wherein the edible composition is a pharmaceutical preparation, and further comprises a pharmaceutically active ingredient.

11. The pharmaceutical preparation of claim 10, wherein the preparation is in the form of a powder.

12. The pharmaceutical preparation of claim 10, wherein the preparation is in the form of a tablet.

13. The pharmaceutical preparation of claim 10, wherein the pharmaceutically active ingredient is acetaminophen.

14. The pharmaceutical preparation of claim 10, wherein the pharmaceutically active ingredient is selected from the group consisting of nourishing and health-promoting agents, antipyretic-analgesic-antiinflammatory agents, antipsychotic drugs, antianxiety drugs, antidepressants, hypnotic-sedatives, spasmolytics, central nervous system affecting drugs, cerebral metabolism ameliolators, antiepileptics, sympathomimetic agents, gastrointestinal function conditioning agents, antacids, antiulcer agents, antitussive-expectorants, antiemetics, respiratory stimulants, bronchodilators, antiallergic agents, dental buccal drugs, antihistamines, cardiotonics, antiarrhythmic agents, diuretics, hypotensive agents, vasoconstrictors, coronary vasodilators, peripheral vasodilators, antihyperlipidemic agents, cholagogues, antibiotics, chemotherapeutic agents, antidiabetic agents, drugs for osteoporosis, skeletal muscle relaxants, antidinics, hormones, alkaloid narcotics, sulfa drugs, antipodagrics, anticoagulants, anti-malignant tumor agents, and treatment agents for Alzheimer's disease.

15. The edible composition of claim 3, wherein the inorganic particles are coated with the stearic compound.

16. The edible composition according to claim 1, wherein the hydrophobization agent is selected from alkaline earth stearates.

17. The edible composition according to claim 1, wherein the hydrophobization agent is a food-grade mineral oil.

18. The edible composition according to claim 1, wherein the conditioner comprises about 1 wt % to about 20 wt %, based on the total weight of the conditioner, of food-grade mineral oil.

19. The pharmaceutical preparation of claim 1, wherein the hydrophobization agent is present in a concentration of about 1 wt % to about 20 wt %, based on the total weight of the conditioner.

20. A pharmaceutical preparation comprising a pharmaceutically active ingredient and a coated conditioner, the conditioner containing inorganic particles and a hydrophobization agent.

21. The pharmaceutical preparation of claim 20, wherein the pharmaceutically active ingredient is acetaminophen.

22. The pharmaceutical preparation of claim 20, wherein the pharmaceutical preparation is in the form of a tablet.

23. The pharmaceutical preparation of claim 20, wherein the hydrophobization agent is present in a concentration of about 1 wt % to about 20 wt %, based on the total weight of the conditioner.

24. The pharmaceutical preparation of claim 20, wherein the hydrophobization agent is selected from the group consisting of food-grade fatty acids, food-grade oils, food-grade waxes, and food-grade gums.

25. The pharmaceutical preparation of claim 20, wherein the hydrophobization agent is a stearic compound and the inorganic particles are coated with the stearic compound.

26. The pharmaceutical preparation of claim 20, wherein the hydrophobization agent is magnesium stearate.

27. The pharmaceutical preparation of claim 20, wherein the hydrophobization agent is food-grade mineral oil.

28. An acetaminophen pharmaceutical preparation comprising:
(a) acetaminophen; and
(b) a coated conditioner comprising:
(i) inorganic particles; and
(ii) 1 wt % to about 20 wt %, based on the total weight of the conditioner, of an hydrophobization agent.

29. The acetaminophen pharmaceutical preparation of claim 28, wherein the preparation in the form of a tablet.
